(19) United States
(12) Patent Application Publication
(10) Pub. No.: US 2024/0165120 A1
(43) Pub. Date: May 23, 2024

(54) TREATING CANCER IN PATIENT HAVING CO-OCCURRING GENETIC ALTERATION IN FGFR2 AND A CANCER DRIVER GENE

(71) Applicants: TAIHO PHARMACEUTICAL CO., LTD., Chiyoda-ku (JP); Karim BENHADJI, Princeton, NJ (US)

(72) Inventors: Karim BENHADJI, Princeton, NJ (US); Volker WACHECK, Princeton, NJ (US); Abdel HALIM, Princeton, NJ (US)

(73) Assignee: TAIHO PHARMACEUTICAL CO., LTD., Chiyoda-ku (JP)

(21) Appl. No.: 18/549,249

(22) PCT Filed: Aug. 30, 2021
(86) PCT No.: PCT/US2021/048206

§ 371 Date: Sep. 6, 2023

Related U.S. Application Data
(60) Provisional application No. 63/158,083, filed on Mar. 8, 2021.

(51) Int. Cl. A61K 31/519; A61K 9/00; A61P 35/00; C12Q 1/6886

(52) U.S. Cl. CPC A61K 31/519; A61K 9/0053; A61P 35/00; C12Q 1/6886; C12Q 2600/156

(57) ABSTRACT

A method of treating a subject with cholangiocarcinoma having a co-occurring genetic alteration in FGFR2 and a cancer driver gene selected from TP53, BAP1, ARID1A, MLL2, PIK3C2B, IKBKE, MCL1, MDM4, and MYC, whereby the subject is administered (S)-1-[(3)-[4-amino-3-[(3,5-dimethoxyphenyl)ethynyl]-1H-pyrazolo[3,4-d]pyrimidin-1-yl]-1-pyrrolidinyl]-2-propen-1-one or a pharmaceutically acceptable salt thereof.

### CROSS REFERENCE TO RELATED APPLICATIONS

This application claims the benefit of priority to U.S. Provisional Application No. 63/158,083 filed Mar. 8, 2021, which is incorporated herein by reference in its entirety.

### BACKGROUND OF THE INVENTION

### Field of the Invention

The present invention relates to treating a cancerous tumor harboring a co-occurring genetic alteration in FGFR2 and a cancer driver gene, such as TP53, BAP1, ARID1A, MLL2, PIK3C2B, IKBKE, MCL1, MDM4, and MYC.

### Description of the Related Art

Cholangiocarcinoma (CCA), a bile duct cancer, is a rare tumor that arises from the malignant transformation of epithelial cells of the bile ducts. It is typically classified as either intrahepatic (iCCA) or extrahepatic (eCCA). Intrahepatic cholangiocarcinoma develops in the smaller bile ducts inside the liver and is the least common form of the disease (approximately 10%), whereas eCCA includes cancers in the peri-hilar (also known as Klatskin tumor) and distal bile duct area and is most common (approximately 90%).

For disease that is localized at diagnosis, surgical resection offers the only chance of cure for patients with CCA. Unfortunately, symptoms are not usually apparent until CCA is at an advanced stage, and thus, most patients (>65%) have disease which is unresectable at diagnosis. Unresectable locally advanced (stage III) and metastatic (stage IV) disease has a poor prognosis with 5-year overall survival (OS) of 10% and 0%, respectively. For such patients, chemotherapy and supportive care are usually offered. See Lamarca A, Hubner R A, Ryder W D, et al. Second-line chemotherapy in advanced biliary cancer: a systematic review. Annals of Oncology. 2014; 25:2328-2338. Gemcitabine-cisplatin is the standard 1st line chemotherapy regimen for patients with advanced, metastatic, unresectable CCA, providing only a modest overall survival of 1 year to patients with advanced iCCA. There is no standard regimen beyond first line treatment. See Valle J, Wasan H. Palmer D H, et al. Cisplatin plus gemcitabine versus gemcitabine for biliary tract cancer. NEJM. 2010; 362:1273-1281. In the second line treatment setting, a retrospective evaluation of 761 patients with advanced biliary tract cancers, including CCA has shown a median overall response rate of 7.7% (95% confidence interval (CI): 5% to 11%) and a median progression-free survival (PFS) of 3.2 months (95% CI: 2.7-3.7 months). Specifically, patients with advanced iCCA have a 6.2 month median overall survival with second-line FOLOX (fluorouracil, leucovorin, and oxaliplatin) therapy. See Lamarca A, Hubner R A, Ryder W D, et al. Second-line chemotherapy in advanced biliary cancer: a systematic review. Annals of Oncology. 2014; 25:2328-2338. These poor results confirm a substantial unmet medical need for new therapies in patients with advanced CCA who have failed initial chemotherapy.

The fibroblast growth factor receptor (FGFR) signaling axis has been well characterized for its role in proliferation, differentiation, migration, and survival, and it is fundamental to embryonic development, regulation of angiogenesis, and wound healing in adults. Dysregulation of the FGFR signaling pathway has been associated with many developmental disorders and with cancer. An extensive amount of literature indicates that FGFR is one of the receptor tyrosine kinases most frequently mutated or otherwise abnormally activated in late-stage human cancer.

Although CCA is known to have the histological and molecular features of an adenocarcinoma of epithelial cells lining the biliary tract, the actual cell of origin is unknown. Fibroblast growth factor/fibroblast growth factor receptor aberrations are a reported genetic modification in CCA. In iCCA, fibroblast growth factor receptor 2 (FGFR2) gene rearrangement including fusions has been identified as an early driver of oncogenic events. These gene rearrangement/fusions are present in an estimated 10% to 20% of patients. See Hanahan D. Weinberg R A. The hallmarks of cancer. Cell. 2000 Jan. 7; 100:57-70; Borad, M. J., Gores, G. J., & Roberts, L. R (2015). Fibroblast growth factor receptor 2 fusions as a target for treating cholangiocarcinoma. Current Opinion in Gastroenterology. 31(3), 264-268; and Goyal L, Saha S, Liu L, et al. Polyclonal Secondary FGFR2 Mutations Drive Acquired Resistance to FGFR Inhibition in Patients with FGFR2 Fusion-Positive Cholangiocarcinoma. Cancer Discov. 2017; 7(3)252-263.

Recently, pemigatinib (PEMAZYRE, Incyte Corporation)—a selective competitive inhibitor of FGFR1, 2, and 3 via inhibition of receptor autophosphorylation—has received U.S. Food and Drug Administration (FDA) approval for the treatment of locally advanced or metastatic cholangiocarcinoma with an FGFR2 fusion or rearrangement in patients who had received prior treatment. Approval was based on results of a clinical trial that enrolled 107 patients with locally advanced or metastatic cholangiocarcinoma with an FGFR2 fusion or rearrangement, in which pemigatinib monotherapy resulted in an independent centrally confirmed objective response rate (ORR) of 35.5% and a median progression-free survival (PFS) of 6.9 (95% CI, 6.2-9.6) months. See Silverman I M, Hollebecque A, Friboulet L, et al. Clinicogenomic Analysis of FGFR2-Rearranged Cholangiocarcinoma Identifies Correlates of Response and Mechanisms of Resistance to Pemigatinib. Cancer Discov February 2021 (11) (2) 326-339.

However, drug resistance, in the form of either gain-of-function alteration of oncogenic driver genes or loss-of-function alterations in tumor suppressor genes, is emerging as a major challenge for FGFR inhibitors. A high percentage (63.0%) of cholangiocarcinoma patients harboring a FGFR2 rearrangement (including fusions) have also been found to have a co-occurring alteration in a well-known tumor suppressor gene including BAP1, CDKN2A/B, TP53, PBRM1, ARID1A, or PTEN, which may provide a mechanism of primary resistance. Patients with tumor suppressor gene loss have been identified as worse responders to FGFR inhibitors, with a significantly shorter median PFS (6.8 months) as compared to those with no co-occurring alteration in a tumor suppressor gene (11.7 months). See Silverman I M, Hollebecque A, Friboulet L, et al. Clinicogenomic Analysis of FGFR2-Rearranged Cholangiocarcinoma Identifies Correlates of Response and Mechanisms of Resistance to Pemigatinib. Cancer Discov February 2021 (11) (2) 326-339. For example, patients with a co-occurring TP53 alteration had no objective response to pemigatinib and a significantly shorter median PFS (2.8 months) compared to those without TP53 gene loss (9.0 months). These results are consistent with previous studies indicating that patients harboring genetic alterations in FGFR2 with co-occurring alterations in tumor suppressor genes, such as TP53, have shorter overall survival (Jain A, Borad M J, Kelley R K, et al. Cholangiocarcinoma with FGFR genetic aberrations: a unique clinical phenotype. JCO Precis Oncol 2018:1-12). Collectively, such literature data indicates that patients with a co-occurring alteration of FGFR2 and certain cancer driver genes, especially in tumor suppressor genes such as BAP1. CDKN2A/B, TP53, PBRM1, ARID1A, or PTEN, are unlikely to respond well to treatment with FGFR inhibitors.

In view of the forgoing, there exists a need for new treatment methods in patients with cholangiocarcinoma harboring a co-occurring genetic alteration in FGFR2 and cancer driver genes.

### SUMMARY OF THE INVENTION

Accordingly, it is an object of the present invention to provide methods of treating subjects with cholangiocarcinoma, in particular intrahepatic cholangiocarcinoma (iCCA), harboring a co-occurring genetic alteration in FGFR2 and a cancer driver gene.

This and other objects, which will become apparent during the following detailed description, have been achieved by the inventors' unexpected discovery that (S)-1-[(3)-[4-amino-3-[(3,5-dimethoxyphenyl)ethynyl]-1H-pyrazolo[3,4-d]pyrimidin-1-yl]-1-pyrrolidinyl]-2-propen-1-one or a pharmaceutically acceptable salt thereof, a pan-FGFR irreversible inhibitor, can be used for treating CCA in subjects with a co-occurring genetic alteration in FGFR2 and one or more of TP53, BAP1, ARID1A, MLL2, PIK3C2B, IKBKE, MCL1, MDM4, and MYC. Thus, the present invention provides:
(1) A method of treating a subject with cholangiocarcinoma having a co-occurring genetic alteration in FGFR2 and a cancer driver gene selected from the group consisting of TP53, BAP1, ARID1A, MLL2, PIK3C2B, IKBKE, MCL1, MDM4, and MYC, the method comprising administering to the subject (S)-1-[(3)-[4-amino-3-[(3,5-dimethoxyphenyl)ethynyl]-1H-pyrazolo[3,4-d]pyrimidin-1-yl]-1-pyrrolidinyl]-2-propen-1-one or a pharmaceutically acceptable salt thereof. This compound (also known as futibatinib) having the formula below is referred to as Compound (1):

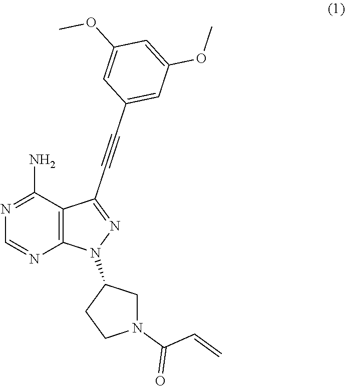 [SMILES: C=CC(=O)N1CC[C@H](n2nc(C#Cc3cc(OC)cc(OC)c3)c3c(N)ncnc32)C1]

(2) The method of (1), wherein the genetic alteration in FGFR2 is a FGFR2 rearrangement or fusion.
(3) The method of (1) or (2), wherein the genetic alteration in FGFR2 is a FGFR2 rearrangement.
(4) The method of (l) or (2), wherein the genetic alteration in FGFR2 is a FGFR2 fusion.
(5) The method of (2) or (4), wherein the FGFR2 fusion is selected from the group consisting of FGFR2-ARHGAP22, FGFR2-AXDND1, FGFR2-AZI1, FGFR2-BEND3, FGFR2-BFSP2, FGFR2-BICC1, FGFR2-CA10, FGFR2-CCDC147, FGFR2-CEP44, FGFR2-CEP55, FGFR2-CIT, FGFR2-CREB5, FGFR2-CTNNA3, FGFR2-CUX1, FGFR2-DDX21, FGFR2-EVI5, FGFR2-GPHN, FGFR2-INA, FGFR2-KIAA1217, FGFR2-KIAA1524, FGFR2-KIAA1598, FGFR2-LRBA, FGFR2-MACF1, FGFR2-MYH9, FGFR2-NRBF2, FGFR2-OFD1, FGFR2-PDE3B, FGFR2-POC1B, FGFR2-PUM1, FGFR2-RBM20, FGFR2-RXRG, FGFR2-SEC21IP, FGFR2-SH3KBP1, FGFR2-SHROOM3, FGFR2-SLMAP, FGFR2-SMARCC1, FGFR2-SORBS1, FGFR2-SYNPO2, FGFR2-TACC1, FGFR2-TACC2, FGFR2-TBC1D4, FGFR2-TRIM8, FGFR2-TUFT1, FGFR2-TXLNA, FGFR2-VCL, and FGFR2-WAC.
(6) The method of any one of (2), (4), or (5), wherein the FGFR2 fusion is selected from the group consisting of FGFR2-ARHGAP22, FGFR2-AXDND1, FGFR2-BEND3, FGFR2-BFSP2, FGFR2-BICC1, FGFR2-CCDC147, FGFR2-CIT, FGFR2-CTNNA3, FGFR2-CIA, FGFR2-DDX21, FGFR2-GPHN, FGFR2-KIAA1217, FGFR2-KIAA1524, FGFR2-KIAA1598, FGFR2-MACF1, FGFR2-PDE3B, FGFR2-RBM20, FGFR2-RXRG, FGFR2-SH3KBP1, FGFR2-SMARCC1, FGFR2-TACC2, FGFR2-TACC2, FGFR2-TUFT1, and FGFR2-VCL.
(7) The method of any one of (2) or (4) to (6), wherein the FGFR2 fusion is selected from the group consisting of FGFR2-BICC1, FGFR2-KIAA1217, and FGFR2-SMARCC1.
(8) The method of any one of (1) to (7), wherein the cancer driver gene is selected from the group consisting of TP53, BAP1, and ARID1A.
(9) The method of any one of (1) to (7), wherein the cancer driver gene is selected from the group consisting of BAP1, ARID1A, MLL2, PIK3C2B, IKBKE, MCL1, MDM4, and MYC.
(10) The method of any one of (1) to (7), wherein the cancer driver gene is selected from the group consisting of BAP1 and ARID1A.
(11) The method of any one of (1) to (7), wherein the cancer driver gene is TP53.
(12) The method of (11), wherein the genetic alteration in TP53 is a short-variant mutation.
(13) The method of any one of (1) to (7), wherein the cancer driver gene is BAP1.
(14) The method of (13), wherein the genetic alteration in BAP1 is a short-variant mutation or a copy-number alteration.
(15) The method of any one of (1) to (7), wherein the cancer driver gene is ARID1A.
(16) The method of (15), wherein the genetic alteration in ARID1A is a short-variant mutation.
(17) The method of any one of (1) to (7), wherein the cancer driver gene is MLL2.
(18) The method of (17), wherein the genetic alteration in MLL2 is a short-variant mutation.
(19) The method of any one of (1) to (7), wherein the cancer driver gene is PIK3C2B.
(20) The method of (19), wherein the genetic alteration in PIK3C2B is a short-variant mutation or a copy-number alteration.
(21) The method of any one of (1) to (7), wherein the cancer driver gene is IKBKE.
(22) The method of (21), wherein the genetic alteration in IKBKE is a short-variant mutation or a copy-number alteration.
(23) The method of any one of (1) to (7), wherein the cancer driver gene is MCL1.
(24) The method of (23), wherein the genetic alteration in MCL1 is a copy-number alteration.
(25) The method of any one of (1) to (7), wherein the cancer driver gene is MDM4.
(26) The method of (25), wherein the genetic alteration in MDM4 is a short-variant mutation or a copy-number alteration.
(27) The method of any one of (1) to (7), wherein the cancer driver gene is MYC.
(28) The method of (27), wherein the genetic alteration in MYC is a copy-number alteration.
(29) The method of any one of (1) to (28), wherein the subject with cholangiocarcinoma is determined to have the co-occurring genetic alteration in FGFR2 and the cancer driver gene prior to the administering.
(30) The method of any one of (1) to (29), wherein the cholangiocarcinoma is intrahepatic cholangiocarcinoma.
(31) The method of any one of (1) to (29), wherein the cholangiocarcinoma is extrahepatic cholangiocarcinoma.
(32) The method of any one of (1) to (31), wherein the cholangiocarcinoma is unresectable.
(33) The method of any one of (1) to (32), wherein the subject with cholangiocarcinoma has previously undergone a chemotherapy regimen prior to the administering.
(34) The method of any one of (1) to (33), wherein the subject with cholangiocarcinoma has previously undergone a chemotherapy regimen with at least one selected from the group consisting of gemcitabine, cisplatin, fluorouracil, leucovorin, and oxaliplatin, prior to the administering.
(35) The method of any one of (1) to (34), wherein the (S)-1-[(3)-[4-amino-3-[(3,5-dimethoxyphenyl)ethynyl]-1H-pyrazolo[3,4-d]pyrimidin-1-yl]-1-pyrrolidinyl]-2-propen-1-one or a pharmaceutically acceptable salt thereof is administered orally to the subject.
(36) The method of any one of (1) to (35), wherein the (S)-1-[(3)-[4-amino-3-[(3,5-dimethoxyphenyl)ethynyl]-1H-pyrazolo[3,4-d]pyrimidin-1-yl]-1-pyrrolidinyl]-2-propen-1-one or a pharmaceutically acceptable salt thereof is administered to the subject once per day (QD).
(37) The method of any one of (1) to (36), wherein 1 to 20 mg of (S)-1-[(3)-[4-amino-3-[(3,5-dimethoxyphenyl)ethynyl]-1H-pyrazolo[3,4-d]pyrimidin-1-yl]-1-pyrrolidinyl]-2-propen-1-one or a pharmaceutically acceptable salt thereof is administered to the subject per day.
(38) The method of any one of (1) to (37), wherein the (S)-1-[(3)-[4-amino-3-[(3,5-dimethoxyphenyl)ethynyl]-1H-pyrazolo[3,4-d]pyrimidin-1-yl]-1-pyrrolidinyl]-2-propen-1-one or a pharmaceutically acceptable salt thereof is administered daily to the subject for at least 21 days.
(39) An antitumor agent for treating a subject with cholangiocarcinoma having a co-occurring genetic alteration in FGFR2 and a cancer driver gene selected from the group consisting of TP53, BAP1, ARID1A, MLL2, PIK3C2B, IKBKE, MCL1, MDM4, and MYC, the antitumor agent comprising (S)-1-[(3)-[4-amino-3-[(3,5-dimethoxyphenyl)ethynyl]-1H-pyrazolo[3,4-d]pyrimidin-1-yl]-1-pyrrolidinyl]-2-propen-1-one or a pharmaceutically acceptable salt thereof
(40) Use of (S)-1-[(3)-[4-amino-3-[(3,5-dimethoxyphenyl)ethynyl]-1H-pyrazolo[3,4-d]pyrimidin-1-yl]-1-pyrrolidinyl]-2-propen-1-one or a pharmaceutically acceptable salt thereof in the treating of a subject with cholangiocarcinoma having a co-occurring genetic alteration in FGFR2 and a cancer driver gene selected from the group consisting of TP53, BAP1, ARID1A, MLL2, PIK3C2B, IKBKE, MCL1, MDM4, and MYC.
(41) A method of treating a subject with cancer having a co-occurring genetic alteration in FGFR2 and a cancer driver gene selected from the group consisting of TP53, BAP1, ARID1A, MLL2, PIK3C2B, IKBKE, MCL1, MDM4, and MYC, the method comprising administering to the subject (S)-1-[(3)-[4-amino-3-[(3,5-dimethoxyphenyl)ethynyl]-1H-pyrazolo[3,4-d]pyrimidin-1-yl]-1-pyrrolidinyl]-2-propen-1-one or a pharmaceutically acceptable salt thereof.
(42) An antitumor agent for treating a subject with cancer having a co-occurring genetic alteration in FGFR2 and a cancer driver gene selected from the group consisting of TP53, BAP1, ARID1A, MLL2, PIK3C2B, IKBKE, MCL1, MDM4, and MYC, the antitumor agent comprising (S)-1-[(3)-[4-amino-3-[(3,5-dimethoxyphenyl)ethynyl]-1H-pyrazolo[3,4-d]pyrimidin-1-yl]-1-pyrrolidinyl]-2-propen-1-one or a pharmaceutically acceptable salt thereof.
(43) Use of (S)-1-[(3)-[4-amino-3-[(3,5-dimethoxyphenyl)ethynyl]-1H-pyrazolo[3,4-d]pyrimidin-1-yl]-1-pyrrolidinyl]-2-propen-1-one or a pharmaceutically acceptable salt thereof in the treating of a subject with cancer having a co-occurring genetic alteration in FGFR2 and a cancer driver gene selected from the group consisting of TP53, BAP1, ARID1A, MLL2, PIK3C2B, IKBKE, MCL1, MDM4, and MYC.
(44) (S)-1-[(3)-[4-amino-3-[(3,5-dimethoxyphenyl)ethynyl]-1H-pyrazolo[3,4-d]pyrimidin-1-yl]-1-pyrrolidinyl]-2-propen-1-one or a pharmaceutically acceptable salt thereof when used for use in treatment of cancer having a co-occurring genetic alteration in FGFR2 and a cancer driver gene selected from the group consisting of TP53. BAP1, ARID1A, MLL2, PIK3C2B, IKBKE, MCL1, MDM4, and MYC.
(45) A pharmaceutical composition comprising (S)-1-[(3)-[4-amino-3-[(3,5-dimethoxyphenyl)ethynyl]-1H-pyrazolo[3,4-d]pyrimidin-1-yl]-1-pyrrolidinyl]-2-propen-1-one or a pharmaceutically acceptable salt thereof for the treatment of cancer having a co-occurring genetic alteration in FGFR2 and a cancer driver gene selected from the group consisting of TP53, BAP1. ARID1A, MLL2, PIK3C2B, IKBKE, MCL1, MDM4, and MYC.

### DETAILED DESCRIPTION OF THE PREFERRED EMBODIMENTS

Compound (1) is a novel, highly selective, potent, and covalently binding irreversible inhibitor of all 4 FGFR isoforms, with half maximal inhibitory concentration (TC50) values (nmol/L) of 3.9 for FGFR1, 1.3 for FGFR2, 1.6 for FGFR3, and 8.3 for FGFR4. In vivo studies show that Compound (1) has strong antitumor efficacy in tumors with various FGFR gene abnormalities, such as FGFR1 or FGFR2 amplification and FGFR3 translocation.

Compound (1) is described in U.S. Pat. Nos. 9,108,973, 10,124,003, US2019/0015417, US2016/0193210, US2019/0183897, U.S. Pat. No. 10,434,103, US2019/0350932, US2021/0030755, WO2019/181876, WO2020/096042, WO2020/110974, WO2020/175697, WO2020/175704, WO2020/256096, and WO2021/153703, the contents of which are incorporated herein by reference in their entirety.

Compound (1) can be used directly or in the form of a pharmaceutically acceptable salt. The phrase “pharmaceutically acceptable” is employed herein to refer to those compounds, materials, compositions, and/or dosage forms which are, within the scope of sound medical judgment, suitable for use in contact with the tissues of human beings without excessive toxicity, irritation, allergic response, or other problem or complication, commensurate with a reasonable benefit/risk ratio. The pharmaceutically acceptable salt of Compound (1) is not particularly limited, and examples thereof include addition salts with inorganic acids such as hydrochloric acid, hydrobromic acid, sulfuric acid, sulfamic acid, phosphoric acid, nitric acid, and the like; organic acids such as acetic acid, propionic acid, succinic acid, glycolic acid, stearic acid, lactic acid, malic acid, tartaric acid, citric acid, ascorbic acid, pamoic acid, maleic acid, hydroxymaleic acid, phenylacetic acid, glutamic acid, benzoic acid, salicylic acid, sulfanilic acid, 2-acetoxybenzoic acid, fumaric acid, toluenesulfonic acid, methanesulfonic acid, ethane disulfonic acid, oxalic acid, isethionic acid, and the like; salts with alkali metals such as potassium, sodium, and the like; salts with alkaline earth metals such as calcium, magnesium, and the like, and salts with organic bases such as ammonium salts, ethylamine salts, alginate, and the like. The pharmaceutically acceptable salts can be synthesized by conventional chemical methods, generally by reacting Compound (1) with a stoichiometric amount of the appropriate base or acid in water or in an organic solvent (e.g., ether, ethyl acetate, ethanol, isopropanol, or acetonitrile), or in a mixture of the two.

Compound (1) or a pharmaceutically acceptable salt thereof may be in the form of a “solvate”, which refers to a physical association of a referenced compound with one or more solvent molecules, whether organic or inorganic. This physical association includes hydrogen bonding. In certain instances, the solvate will be capable of isolation, for example when one or more solvent molecules are incorporated in the crystal lattice of the crystalline solid. The solvent molecules in the solvate may be present in a regular arrangement and/or a non-ordered arrangement. The solvate may comprise either a stoichiometric or nonstoichiometric amount of the solvent molecules. Solvate encompasses both solution phase and isolable solvates. Exemplary solvent molecules which may form the solvate include, but are not limited to, water, methanol, ethanol, n-propanol, isopropanol, n-butanol, isobutanol, tert-butanol, ethyl acetate, glycerin, acetone, and the like.

Compound (1) can exist in a crystal form that exhibits an X-ray powder diffraction spectrum containing at least three characteristic peaks at diffraction angles (20±0.2°) selected from 9.5°, 14.3°, 16.7°, 19.1°, 20.8°, 21.9°, and 25.2°. Compound (1) can exist in a crystal form that exhibits an X-ray powder diffraction spectrum containing at least seven characteristic peaks at diffraction angles (20±0.2°) selected from 13.5°, 17.9°, 19.5°, 20.6°, 22.0°, 22.6°, 23.3°, 23.7°, and 24.2°. A crystal meeting either of these criteria shows good stability, excellent oral absorbability, high chemical purity, and is suitable for mass production. Methods for preparing such crystal forms are described in U.S. Pat. No. 10,434,103, the contents of which are incorporated herein by reference in its entirety.

The terms “treat”, “treating”, or the “treatment” of cancers in the present disclosure includes any effect, e.g., lessening, reducing, modulating, stabilizing, ameliorating or eliminating, that results in the improvement of the condition, disease, disorder, and the like, or ameliorating a symptom thereof. Specifically, these terms may refer to: (1) a stabilization, reduction (e.g., by more than 10%, 20%, 30%, 40%, 50%, preferably by more than 60% of the population of cancer cells and/or tumor size as compared to prior to administration), or elimination of the cancer cells, (2) inhibiting cancerous cell division and/or cancerous cell proliferation, (3) relieving to some extent (or, preferably, eliminating) one or more symptoms associated with a pathology related to or caused in part by unregulated or aberrant cellular division, (4) an increase in disease-free, relapse-free, progression-free, and/or overall survival, duration, or rate, (5) a decrease in hospitalization rate, (6) a decrease in hospitalization length, (7) eradication, removal, or control of primary, regional and/or metastatic cancer, (8) a stabilization or reduction (e.g., by at least 10%, 20%, 30%, 40%, 50%, 60%, 70%, preferably at least 80% relative to the initial growth rate) in the growth of a tumor or neoplasm, (9) an impairment in the formation of a tumor, (10) a reduction in mortality. (11) an increase in the response rate, the durability of response, or number of patients who respond or are in remission, (12) the size of the tumor is maintained and does not increase or increases by less than 10%, preferably less than 5%, preferably less than 4%, preferably less than 2%, (13) a decrease in the need for surgery (e.g., colectomy, mastectomy), and/or (14) preventing or reducing the metastasis of cancer cells.

Genetic alterations of fibroblast growth factor receptor (FGFR) are tumorigenic drivers that exist across different tumor types, with genetic alterations having been observed in all FGFR subtypes (FGFR1, FGFR2, FGFR3, and FGFR4). The cancers treated herein are those having a genetic alteration of FGFR, in particular FGFR2, and a co-occurring genetic alteration in at least one cancer driver gene (other than FGFR).

“Cancer driver genes” are genes that give cells a growth advantage when they are genetically altered, helping tumors proliferate. Cancer driver genes generally fall into two classes: tumor suppressor genes and oncogenes. “Tumor suppressor genes”, or anti-oncogenes, provide negative control of cell proliferation. Loss-of-function of the proteins encoded by these genes, through deletion or inactivation of the gene, liberates the cell from growth constraints and contributes to malignant transformation. “Oncogenes” are genes that normally help cells grow, that when genetically altered result in activated or over-expressed levels of proteins that can cause those cells designated for apoptosis to survive and proliferate instead. Thus, the gain-of-function of oncogenes together with the loss-of-function of tumor suppressor genes determine the processes that control tumor formation and development.

In the present disclosure, a “genetic alteration” includes gene amplification (e.g., copy-number alterations), gene mutation, chromosomal translocation/insertion/inversion, gene rearrangement or gene fusion (a subset of gene rearrangements), and the like.

The cancers which can be treated include, but are not limited to, cholangiocarcinoma, breast cancer, colorectal cancer, brain tumors, urothelial cancer, head and neck cancers, esophageal cancer, cervical cancer, gastric cancer, non-small cell lung cancer, sarcomas, skin cancer, appendix cancer, endometrial cancer, gallbladder cancer, mesothelioma, neuroendocrine tumors, neuroblastoma, ovarian cancer, prostate cancer, renal cell carcinoma, and myeloid-lymphoid neoplasms. The methods disclosed herein may also be used as a tumor-agnostic treatment for malignancies having the co-occurring genetic alteration. The cancers treated are usually solid cancers. While cancers at various stages and resectabilities may respond to the disclosed treatment, the methods herein may be particularly useful in the treatment of unresectable, locally advanced (stage III) and metastatic (stage IV) disease.

The treatment methods of the present disclosure are particularly useful in the treatment of cholangiocarcinoma, including both iCCA and eCCA, with particular preference given to iCCA. Subjects with risk factors for cholangiocarcinoma include those with primary sclerosing cholangitis, ulcerative colitis, cirrhosis, hepatitis C, hepatitis B, infection with certain liver flukes, and some congenital liver malformations. However, many people have no identifiable risk factors.

Subjects harboring one or more genetic alterations of FGFR, in particular those with FGFR2 alterations, are candidates for treatment herein. FGFR2 alterations are key oncogenic drivers that cause constitutive FGFR2 signaling, which in turn contributes to a variety of tumorigenic processes.

The genetic alteration of FGFR2 may be in the form of a rearrangement. FGFR2 “rearrangements” include those with a genomic breakpoint within the FGFR2 intron 17 or exon 18 hotspot and with either (i) a novel partner gene predicted to be out of frame or out of strand with FGFR2, or (ii) no identifiable partner gene.

The genetic alteration of FGFR2 may be in the form of a fusion. FGFR2 rearrangements are further defined as “fusions” (i) if the genomic breakpoint is within the intron 17 or exon 18 hotspot and (ii) if the fusion gene partner is either a previously described fusion partner or a novel gene partner predicted to be an in-frame fusion with FGFR2. Therefore, FGFR2 fusions are considered herein to be a subset of FGFR2 rearrangements. Advantageously, there is no significant difference in objective response rate (ORR) for subjects with FGFR2 fusions versus those with an FGFR2 rearrangements in the methods herein.

FGFR2 fusions may be formed from various fusion partners (listed below as Xin FGFR2-X), the selection of fusion partner is not particularly limiting. Examples of FGFR2 fusions include, but are not limited to, FGFR2-ARHGAP22, FGFR2-AXDND1, FGFR2-AZI1, FGFR2-BEND3, FGFR2-BFSP2, FGFR2-BICC1, FGFR2-C4A10, FGFR2-CCDC147, FGFR2-CEP44, FGFR2-CEP55, FGFR2-CIT, FGFR2-CREB5, FGFR2-CTNNA3, FGFR2-CUX1, FGFR2-DDX21, FGFR2-EVI5, FGFR2-GPHN, FGFR2-INA, FGFR2-KIAA1217, FGFR2-KIAA1524, FGFR2-KIAA1598, FGFR2-LRBA, FGFR2-MACF1, FGFR2-MYH9, FGFR2-NRBF2, FGFR2-OFD1, FGFR2-PDE3B, FGFR2-POC1B, FGFR2-PUM1, FGFR2-RBM20, FGFR2-RXRG, FGFR2-SEC21IP, FGFR2-SH3KBP1, FGFR2-SHROOM3, FGFR2-SLMAP, FGFR2-SMARCC1, FGFR2-SORBS1, FGFR2-SYNPO2, FGFR2-TACC1, FGFR2-TACC2, FGFR2-TBC1D4, FGFR2-TRIM8, FGFR2-TUFT1, FGFR2-TXLNA, FGFR2-VCL, and FGFR2-WAC.

Based on predicted response rate and relative frequency, preferred subjects are those harboring FGFR2 fusions of FGFR2-ARHGAP22, FGFR2-AXDND1, FGFR2-BEND3, FGFR2-BFSP2, FGFR2-BICC1, FGFR2-CCDC147, FGFR2-CIT, FGFR2-CTNNA3, FGFR2-CUX1, FGFR2-DDX21, FGFR2-GPHN, FGFR2-KIAA1217, FGFR2-KIAA1524, FGFR2-KIAA1598, FGFR2-MACF1, FGFR2-PDE3B, FGFR2-RBM20, FGFR2-RXRG, FGFR2-SH3KBP1, FGFR2-SMARCC1, FGFR2-TACC1, FGFR2-TACC2, FGFR2-TUFT1, and FGFR2-VCL, with particular preference given to FGFR2-BICC1, FGFR2-KIAA1217, and FGFR2-SMARCC1.

The presence of FGFR2 fusions or rearrangements may be determined. e.g., during subject pre-screening or from previous testing performed on the subject, or otherwise confirmed according to known assays, including FDA approved diagnostic/prognostic assays. Examples of which include, but are not limited to, testing by Foundation Medicine (e.g., FoundationOne™ CDx assay), testing by Sysmex Corporation (e.g., OncoGuide™ NCC Oncopanel System), next generation sequencing (NGS), fluorescence in situ hybridization (FISH), or other assays that can determine FGFR2 gene fusions or other FGFR2 rearrangements on tumor tissues or from ctDNA. For example, subjects which do not have archival tumor tissue samples can be biopsied and the fresh tumor biopsy can be analyzed or submitted. e.g., to Foundation Medicine, for confirmation of FGFR2 gene fusion or other FGFR2 rearrangements.

Subjects with a genetic alteration of FGFR2 in the form of FGFR2 mutations, such as short-variant mutations, may also be treated by the disclosed methods. The FGFR2 mutations are not required to, but may correspond to a characteristic “gatekeeper” amino acid residue in the kinase and/or be associated with tumors which have become resistant to conventional FGFR inhibitors such as pemigatinib, ponatinib, regorafenib, intedanib, dovitinib lactate, lenvatinib mesylate, cediranib, oratinib, brivanib alaninate, AZD4547, NVP-BGJ398 (infigratinib), sulfatinib, lenvatinib, JNJ-42756493 (erdafitinib), ARQ-087 (derazantinib), S-49076, IMCA1, PRO001, R3Mab, and the like. Examples of FGFR2 mutations include, but are not limited to, mutations of at least one of N550, V565, E566, and K660 of FGFR2, which are described in U.S. Pat. No. 10,124,003 and US2019/0015417, the contents of which are incorporated herein by reference in their entirety.

Subjects which can be treated with Compound (1) or its pharmaceutically acceptable salt also harbor a co-occurring genetic alteration of a cancer driver gene, that is, an alteration of a cancer driver gene in addition to the genetic alteration of FGFR, in particular FGFR2, such as FGFR2 gene fusions/rearrangements described heretofore. Subjects with a co-occurring genetic alteration in the following cancer driver genes have been identified as positive clinical responders to treatment herein: TP53, BAP1, ARID1A, MLL2, PIK3C2B, IKBKE, MCL1, MDM4, and MYC. Treatment may be performed on subjects having one genetic alteration to a cancer driver gene, or more than one genetic alteration to a cancer driver gene. The co-occurring genetic alteration may be to a tumor suppressor gene (i.e., TP53, BAP1, ARID1A, and/or MLL2) or to an oncogene (e.g., PIK3C2B, IKBKE, MCL1, MDM4, and/or MYC).

For example, subjects may have a co-occurring genetic alteration in FGFR2 and TP53. The TP53 gene is a tumor suppressor gene that encodes tumor protein p53, a protein which contains transcriptional activation, DNA binding, and oligomerization domains. The encoded protein responds to diverse cellular stresses to regulate expression of target genes, thereby inducing cell cycle arrest, apoptosis, senescence. DNA repair, or changes in metabolism. Mutations in this gene are associated with a variety of human cancers, and may be of either the germline or somatic variety. The genetic alteration of TP53 may include, but is not limited to, mutations, such as short-variant mutations. The mutation may be of the missense variety.

In another example, subjects may have a co-occurring genetic alteration in FGFR2 and BAP1. The BAP1 gene is a tumor suppressor gene that provides instructions for making the ubiquitin carboxyl-terminal hydrolase BAP1 protein (shortened to BAP1), which functions as a deubiquitinase to regulate cell growth, cell proliferation, and cell death. The genetic alteration of BAP1 may include, but is not limited to, mutations, amplifications, and rearrangements, with particular mention being made to mutations and amplifications such as short-variant mutations or copy-number alterations.

In another example, subjects may have a co-occurring genetic alteration in FGFR2 and ARID1A. The AT-rich interactive domain-containing protein 1A (ARID1A) gene is a tumor suppressor gene that provides instructions for making a protein that forms one subunit of SWI/SNF protein complexes, which regulate gene expression by chromatin remodeling to repair damaged DNA, replicate DNA, and control the growth, division, and differentiation of cells. The genetic alteration of ARID1A may include, but is not limited to, mutations, such as short-variant mutations.

In another example, subjects may have a co-occurring genetic alteration in FGFR2 and MLL2. MLL2 is a tumor suppressor gene encoding histone H3 lysine 4 (H3K4) mono-methyltransferase, that colocalizes with lineage determining transcription factors on transcriptional enhancers and is essential for cell differentiation and embryonic development. The genetic alteration of MLL2 may include, but is not limited to, mutations, such as short-variant mutations.

In another example, subjects may have a co-occurring genetic alteration in FGFR2 and PIK3C2B. Phosphatidylinositol-4-phosphate 3-kinase, catalytic subunit type 2 beta (PIK3C2B) is an oncogene that encodes a phosphoinositide 3-kinase (PI3K) family protein that plays a role in signaling pathways involved in cell proliferation, oncogenic transformation, cell survival, cell migration, and intracellular protein trafficking. The genetic alteration of PIK3C2B may include, but is not limited to, mutations, amplifications, and rearrangements, with particular mention being made to mutations and amplifications such as short-variant mutations or copy-number alterations.

In another example, subjects may have a co-occurring genetic alteration in FGFR2 and IKBKE. As an oncogene, the IKBKE gene encodes the protein IKBKE (inhibitor of nuclear factor kappa-B kinase subunit epsilon), a member of the noncanonical IKK family that is essential in the regulation of inflammatory reactions, activation and proliferation of immune cells, and metabolic diseases. IKBKE shows oncogenic activity through phosphorylation of important signaling targets such as AKT, ERα, and through NFκB activation. The genetic alteration of IKBKE may include, but is not limited to, mutations and amplifications, such as short-variant mutations or copy-number alterations.

In another example, subjects may have a co-occurring genetic alteration in FGFR2 and MCL1. The MCL1 gene is an oncogene that encodes the myeloid cell leukemia 1 (MCL1) protein, which is a potent multidomain anti-apoptotic protein of the BCL2 family that heterodimerizes with other BCL2 family members to protect against apoptotic cell death. The genetic alteration of MCL1 may include, but is not limited to, amplifications, such as copy-number alterations.

In another example, subjects may have a co-occurring genetic alteration in FGFR2 and MDM4. As an oncogene, the MDM4 gene encodes the nuclear Mouse double Minute 4 (MDM4) protein that contains a p53 binding domain at the N-terminus and a RING finger domain at the C-terminus, and shows structural similarity to p53-binding protein MDM2. Both proteins bind the p53 tumor suppressor protein and inhibit its activity, and have been shown to be overexpressed in a variety of human cancers. The genetic alteration of MDM4 may include, but is not limited to, mutations and amplifications, such as short-variant mutations or copy-number alterations.

In yet another example, subjects may have a co-occurring genetic alteration in FGFR2 and MYC. The MYC gene is a proto-oncogene that encodes a nuclear phosphoprotein that plays a role in cell cycle progression, apoptosis and cellular transformation. The encoded protein forms a heterodimer with the related transcription factor MAX. This complex binds to the E box DNA consensus sequence and regulates the transcription of specific target genes. Amplification of this gene is frequently observed in numerous human cancers. The genetic alteration of MYC may include, but is not limited to, amplifications, such as copy-number alterations.

The presence of genetic alterations in cancer driver genes may be determined, e.g., during subject pre-screening or from previous testing performed on the subject, or otherwise confirmed according to known assays, including FDA approved diagnostic/prognostic assays. Examples of which include, but are not limited to, testing by Foundation Medicine (e.g., FoundationOne CDx assay), testing by Sysmex Corporation (e.g., OncoGuide™ NCC Oncopanel System), next generation sequencing (NGS), fluorescence in situ hybridization (FISH), or other assays that can determine gene alterations on tumor tissues or from ctDNA. For example, subjects which do not have archival tumor tissue samples can be biopsied and the fresh tumor biopsy can be analyzed or submitted, e.g., to Foundation Medicine, for confirmation of genetic alterations of the above-identified cancer driver genes.

Like many inhibitor classes, FGFR inhibitors have proven susceptible to resistance mechanisms involving alterations of various cancer driver genes. For instance, patients with co-occurring genetic alterations to FGFR2 and tumor suppressor genes have been found to respond worse overall to treatment with FGFR inhibitors such as pemigatinib compared to patients with unaltered tumor suppressor genes. See Silverman I M, Hollebecque A, Friboulet L, et al. Clinicogenomic Analysis of FGFR2-Rearranged Cholangiocarcinoma Identifies Correlates of Response and Mechanisms of Resistance to Pemigatinib. Cancer Discov February 2021 (11) (2) 326-339. Furthermore, alterations in specific cancer driver genes, such as TP53, have been found to be particularly problematic for the FGFR inhibitor class. For example, patients with a co-occurring TP53 alteration had no objective response to pemigatinib and a significantly shorter median PFS (2.8 months) compared to those without TP53 alterations (9.0 months).

The inventors have unexpectedly found that subjects harboring co-occurring genetic alterations of FGFR2 and a cancer driver gene identified above are responsive to treatment with the FGFR inhibitor of the present disclosure, Compound (1) or its pharmaceutically acceptable salt—with no obvious or significant differences in ORR and PFS being observed in subjects with the altered cancer driver gene compared to subjects in which those cancer driver genes are unaltered. This includes the particular problematic TP53 gene. Therefore, a preferred embodiment of the present disclosure involves treating a subject with cholangiocarcinoma having a co-occurring genetic alteration of FGFR2 and TP53 with Compound (1) or a pharmaceutically acceptable salt thereof. That subjects with a genetic alteration in both FGFR2 and a cancer driver gene selected from TP53, BAP1, ARID1A, MLL2, PIK3C2B, IKBKE, MCL1, MDM4, and MYC, and particularly those genes of the tumor suppressor variety, can be treated with Compound (1) is unexpected in view of previous findings with FGFR inhibitors.

Before commencing treatment, determination may be made as to whether the subject has the co-occurring genetic alterations of FGFR2 and a cancer driver gene identified above. Thus, the methods may involve a pre-screening step to determine whether the subject has the co-occurring genetic alterations and is a good candidate for treatment. The genetic alterations may be determined from family history of cancers involving the genetic alterations, by genotyping the subject or analyzing any tissue sample from the subject including blood or tumor samples taken from the subject using assays such as those described heretofore, or from historical records or previous testing performed on the subject. If the subject has both a genetic alteration in FGFR (e.g., FGFR2) and a genetic alteration in at least one cancer diver gene, treatment with Compound (1) or its pharmaceutically acceptable salt is appropriate.

The terms “administer”, “administering”. “administration”, and the like, refer to the methods that may be used to enable delivery of the active ingredient to the desired site of biological action. Routes or modes of administration are as set forth herein. These methods include, but are not limited to, oral routes, intraduodenal routes, parenteral injection (including intravenous, subcutaneous, intraperitoneal, intramuscular, intravascular, or infusion), topical/transdermal, and rectal/vaginal administration. Those of ordinary skill in the art are familiar with administration techniques that can be employed. Oral administration is preferred.

In the present application, the term “administration schedule” is a plan in which the type, amount, period, procedure, etc. of the drug in the drug treatment are shown in time series, and the dosage, administration method, administration order, administration date, and the like of each drug are indicated. The date specified to be administered is determined before the start of the drug administration. The administration is continued by repeating the course with the set of administration schedules as “courses”.

Regarding the administration schedule of the present invention. “continuous” means administration every day without interruption during the treatment course. If the administration schedule follows an “intermittent” administration schedule, then days of administration may be followed by “rest days” or days of non-administration of drug within the course.

A “drug holiday” indicates that the drug is not administered in a predetermined administration schedule. For example, after undergoing several courses of treatment, a subject may be prescribed a regulated drug holiday as part of the administration schedule, e.g., prior to re-recommencing active treatment.

The dosage amount and treatment duration are dependent on factors, such as bioavailability of a drug, administration mode, toxicity of a drug, gender, age, lifestyle, body weight, the use of other drugs and dietary supplements, the disease stage, tolerance and resistance of the body to the administered drug, etc., and then determined and adjusted accordingly. An appropriate dosage amount may differ from one individual to another. An appropriate dosage amount in any individual case may be determined using techniques, such as dose escalation.

The subject having a genetic alteration of FGFR, in particular FGFR2, and a co-occurring genetic alteration in at least one cancer driver gene can be treated with Compound (1) or its pharmaceutically acceptable salt at dose levels for continuous (7 days of administration in a week) dosing of from about 1 mg/day, from about 2 mg/day, from about 4 mg/day, from about 6 mg/day, from about 8 mg/day, from about 10 mg/day, from about 12 mg/day, from about 14 mg/day, from about 16 mg/day, from about 18 mg/day, and up to about 50 mg/day, up to about 45 mg/day, up to about 40 mg/day, up to about 35 mg/day, up to about 30 mg/day, up to about 25 mg/day, up to about 20 mg/day. The dosing level may be varied within the ranges such as from about 1 mg/day to about 50 mg/day, from about 12 mg/day to about 20 mg/day, and from about 16 mg to about 20 mg/day.

The subject having a genetic alteration of FGFR, in particular FGFR2, and a co-occurring genetic alteration in at least one cancer driver gene can be treated with Compound (1) or its pharmaceutically acceptable salt at dose levels for intermittent dosing of from about 50 mg/day, from about 56 mg/day, from about 60 mg/day, from about 80 mg/day, from about 100 mg/day, from about 120 mg/day, from about 140 mg/day, and up to about 200 mg/day, up to about 190 mg/day, up to about 180 mg/day, up to about 170 mg/day. The dosing level may be varied within the ranges such as from about 50 mg/day to about 200 mg/day, from about 100 mg/day to about 160 mg/day, and from about 120 mg to about 160 mg/day.

The dosing can be continuous (7 days of administration in a week) or intermittent, for example, depending the pharmacokinetics and a particular patients clearance/accumulation of the drug. If intermittently, the schedule may be, for example, 4 days of administration and 3 days off (rest days) in a week or any other intermittent dosing schedule deemed appropriate using sound medical judgement. Continuous administration is preferred. The dosing can be performed once per day (QD) or more than once per day (b.i.d., t.i.d., etc.), with doses of about 12 to 20 mg/day QD being preferred. The daily dose may be administered as a single dose or multiple individual divided doses. For example, five (5) tablets, each tablet containing 4 mg of Compound (1) or its pharmaceutically acceptable salt, may be administered to the subject once per day (QD) for a total dose of 20 mg/day.

The dosing whether continuous or intermittent is continued for a particular treatment cycle, typically at least a 21 day cycle, which can be repeated with or without a drug holiday. Longer or shorter cycles can also be used such as 14 days, 18 days, 24 days, 28 days, 35 days, or any range therebetween. The cycle may be repeated without a drug holiday or with a drug holiday depending upon the subject. Other schedules are possible depending upon the presence or absence of adverse events, response of the cancer to the treatment, patient convenience, and the like. An “adverse event” refers to any unfavorable or unintended illness or symptom thereof occurring in a patient to whom a drug has been administered. It does not matter whether there is a causal relationship with the drug or not. For example, the intermittent dosing can be performed on day 1, 3, 5, 7, 9, 11, 13, 15, 17, 19, and 21; on day 1, 4, 8, 11, 15, and 19; on day 1, 3, 5, 8, 10, 12, 15, 17 and 19 in a 21 day cycle.

The larger doses are usually given intermittently with doses up to about 20 mg usually given continuously (daily). A subject may be started with a low dose and then have the dose escalated until either maximum dose is reached or the subject experiences adverse events at which point the escalation is stopped and the drug dosing reduced to a previous dose where the adverse event was not experienced or was not serious enough to require stoppage of the treatment. A subject that experiences an adverse event may also be managed with dosing interruptions (e.g., a drug holiday), if deemed appropriate. Typical dosing for the continuous regimen may be 12, 16, or 20 mg/day but higher or lower doses may be used depending on the subject's response to the treatment and presence or absence of adverse events. If a dose is well-tolerated, the dose can be increased. The continuous administration may be continued for one cycle, e.g., 21 days, the cycle may then be repeated, as desired.

Such continuous or intermittent administration is applicable also to combination therapies where Compound (1) or its pharmaceutical acceptable salt is administered in combination with one or more other anticancer agents.

The treatment methods of the present disclosure may involve administration of Compound (1) or pharmaceutically acceptable salt thereof as a stand-alone therapy. The treatment may also involve administration as a post-operative auxiliary chemotherapy that is performed to prevent recurrence of tumors after surgically removing tumors, as well as pre-operative auxiliary chemotherapy prior to surgery to surgically remove tumors. In some cases, such as with cholangiocarcinoma, surgery may include a liver transplantation. The treatment may also include administration of Compound (1) or pharmaceutically acceptable salt thereof during or after radiation therapy or as an adjuvant therapy to prevent recurrence of the tumor in a patient where other treatments such as surgery have rendered the patient cancer-free.

Subjects may be treated whom have not previously undergone a chemotherapy regimen, i.e., Compound (1) or its pharmaceutically acceptable salt are administered as first-line chemotherapy. Alternatively, subjects may be treated whom have previously undergone a chemotherapy regimen, i.e., Compound (1) or its pharmaceutically acceptable salt are administered as second-, third-, fourth-, etc. line therapy. A prior chemotherapy regimen may have been performed with a variety of anticancer agents, examples of such anticancer agents will be discussed hereinafter. In the specific case of treating cholangiocarcinoma, notable examples of anticancer agents which may have been administered to the subject in a previous chemotherapy regimen include, but are not limited to, one or more of gemcitabine, cisplatin, fluorouracil, leucovorin, and oxaliplatin, with standard first-line treatment for cholangiocarcinoma being gemcitabine-cisplatin chemotherapy, and standard second-line treatment for cholangiocarcinoma being FOLFOX (fluorouracil-leucovorin-oxaliplatin) chemotherapy.

Subjects may be treated whom have not previously undergone a chemotherapy regimen with FGFR inhibitor(s). Alternatively, subjects may be treated whom have been previously treated with FGFR inhibitor(s), including those conventional FGFR inhibitors described previously.

As described below, Compound (1) or its pharmaceutically acceptable salt may be specially formulated for administration in solid or liquid form, including those adapted for the following. (1) oral administration, for example, drenches (aqueous or non-aqueous solutions or suspensions), tablets or capsules, e.g., those targeted for buccal, sublingual, and systemic absorption, boluses, powders, granules, syrups, pastes for application to the tongue; (2) parenteral administration, for example, by subcutaneous, intramuscular, intravenous or epidural injection as, for example, a sterile solution or suspension, or sustained release formulation; (3) topical application/transdermal administration, for example, as a cream, ointment, or a controlled release patch or spray applied to the skin; (4) intravaginally or intrarectally, for example, as a pessary, cream or foam; or (5) nasally. In the case of Compound (1) or its pharmaceutically acceptable salt, an oral formulation is preferable.

Formulations can be prepared using a pharmaceutically acceptable carrier or the like by using known formulation methods. For example, as a granulation method, a fluidized bed granulation method, a stirring granulation method, a tumbling fluid granulation method, an extrusion granulation method, or the like, can be used. Formulations of Compound (1) are disclosed in US2021/0030755 and WO2019/181876, the contents of which are incorporated herein by reference in their entirety.

Pharmaceutically acceptable carriers are those materials, compositions, or vehicles, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the subject. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations, such as cyclodextrins, liposomes, and micelle forming agents, e.g., bile acids, just to name a few.

Pharmaceutically acceptable carriers may be categorized as various general-purpose agents such as excipients, binders, disintegrating agents, lubricants, diluents, dissolution aids, suspending agents, swelling agents, isotonic agents, pH adjusters, buffers, stabilizers, colorants, flavoring agents, and the like.

Examples of excipients include, but are not limited to, lactose, sucrose, D-mannitol, glucose, starch (corn starch), calcium carbonate, kaolin, microcrystalline cellulose, and silicic acid anhydride.

Examples of binders include, but are not limited to, water, ethanol, 1-propanol, 2-propanol, simple syrup, liquid glucose, liquid a-starch, liquid gelatin, D-mannitol, carboxymethyl cellulose, hydroxypropyl cellulose (e.g., low viscosity hydroxypropyl cellulose), hypromellose, hydroxypropyl starch, methyl cellulose, ethyl cellulose, shellac, calcium phosphate, polyvinylpyrrolidone.

Examples of disintegrants include, but are not limited to, low-substituted hydroxypropylcellulose, dry starch, partially pregelatinized starch, crystalline cellulose, carmellose sodium, carmellose calcium, D-mannitol, crospovidone, sodium alginate, agar powder, sodium hydrogen carbonate, calcium carbonate, sodium lauryl sulfate, stearic acid monoglyceride, and lactose.

Examples of lubricants include, but are not limited to, hydrogenated oil, sucrose fatty acid ester, sodium lauryl sulfate, stearic acid, purified talc, sodium stearate, magnesium stearate, borax, and polyethylene glycol.

Examples of colorants include, but are not limited to, edible yellow No. 5 dye, edible blue No. 2 dye, edible lake dye, iron sesquioxide, yellow sesquioxide, and titanium oxide.

Examples of sweetening/flavoring agents include, but are not limited to, aspartame, saccharin (as sodium, potassium or calcium saccharin), cyclamate (as a sodium, potassium or calcium salt), sucralose, acesulfame-K, thaumatin, neohisperidin, dihydrochalcone, ammoniated glycyrrhizin, dextrose, maltodextrin, fructose, levulose, sucrose, glucose, wild orange peel, citric acid, tartaric acid, oil of wintergreen, oil of peppermint, oil of spearmint, oil of sassafras, oil of clove, cinnamon, anethole, menthol, thymol, eugenol, eucalyptol, lemon, lime, and lemon-lime.

If desired, an enteric coating or a coating to increase the persistence of effects can be provided by methods desirable for oral preparations. Examples of such coating agents include hydroxypropyl methylcellulose, ethyl cellulose, hydroxymethyl cellulose, hydroxypropyl cellulose, polyethylene glycol, and Tween 80 (registered trademark).

Compound (1) or its pharmaceutically acceptable salt are preferably formulated in solid dosage form for oral administration, such as in the form of capsules, tablets, pills, dragees, powders, granules, troches, and the like, with preference given to film-coated tablets. Compound (1) or its pharmaceutically acceptable salt may be mixed with one or more pharmaceutically acceptable carriers such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, poly vinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants (e.g., fatty acid esters of sorbitan and polyalkolyated fatty acid esters of sorbitan such as Tween 80 (registered trademark); (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the formulations may also comprise buffering agents. Solid compositions of a similar type may also be employed as fillers in soft and hard shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface active or dispersing agent. Molded tablets may be made by molding in a suitable machine a mixture of the powdered compound moistened with an inert liquid diluent. The tablets, and other solid dosage forms may optionally be scored or prepared with coatings and shells, such as enteric coatings and other coatings well known in the pharmaceutical formulating art. One example coating formulation may include hypromellose, polyethylene glycol, titanium oxide, and a coloring agent. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria retaining filter, or by incorporating sterilizing agents in the form of sterile solid compositions which can be dissolved in sterile water, or some other sterile injectable medium immediately before use. These formulations may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above described excipients.

Compound (1) or its pharmaceutically acceptable salt can be combined with one or more other anticancer agents, such as those described in US2019/0350932, the disclosure of which is incorporated herein by reference. The anticancer agent is not particularly limited, and examples thereof include antimetabolites (purine antimetabolites, antifolates, and pyrimidine antimetabolites), alkaloid antitumor agents, platinum-containing drugs, molecular targeting drugs (low-molecular-weight molecular targeting drugs, antibody molecular targeting drugs, and immune checkpoint inhibitors), antitumor antibiotics, and alkylating agents.

Examples of antimetabolites include, but are not limited to, purine antimetabolites such as fludarabine, cladribine, and nelarabine; pyrimidine antimetabolites such as 5-fluorouracil (5-FU), tegafur/gimeracil/oteracil potassium, tegafur/uracil, trifluridine/tipiracil hydrochloride, capecitabine, doxifluridine, 5-fluoro-2′-deoxyuridine, gemcitabine, and cytarabine; and antifolates such as pemetrexed and methotrexate.

Examples of alkaloid antitumor agents include, but are not limited to, paclitaxel (including derivatives such as albumin-bound paclitaxel (e.g., ABI-007) and PEG-bound paclitaxel), docetaxel, cabazitaxel, eribulin, irinotecan, nogitecan, etoposide, vinorelbine, vincristine, and vinblastine.

Examples of platinum-containing drugs include, but are not limited to, cisplatin, carboplatin, oxaliplatin, and nedaplatin.

Examples of molecular targeting drugs include, but are not limited to, low-molecular-weight molecular targeting drugs such as imatinib, gefitinib, erlotinib, lapatinib, sunitinib, dasatinib, everolimus, temsirolimus, selumetinib, trametinib, sorafenib, afatinib, regorafenib, dabrafenib, vemurafenib, trans-3-amino-1-methyl-3-(4-(3-phenyl-5H-imidazo[1,2-c]pyrido[3,4-e][1,3]oxazin-2-yl)phenyl)cyclobutanol and pharmaceutically acceptable salts thereof, and 8-[4-(1-aminocyclobutyl)phenyl]-9-phenyl-1,2,4-triazolo[3,4-f][1,6]naphthyridin-3(2H)-one (MK2206) and pharmaceutically acceptable salts thereof, in particular those which target EGFR, MAPK, PI3K/AKT/mTOR, and NFκB signaling pathways; antibody molecular targeting drugs such as trastuzumab, cetuximab, bevacizumab, panitumumab, veltuzumab, rituximab, and ramucirumab; and immune checkpoint inhibitors such as nivolumab, pembrolizumab, atezolizumab, durvalumab, avelumab, ipilimumab, tremelimumab, and abatacept.

Examples of antitumor antibiotics include, but are not limited to, doxorubicin, daunorubicin, epirubicin, actinomycin D. and mitomycin C.

Examples of alkylating agents include, but are not limited to, cyclophosphamide, dacarbazine, temozolomide, nimustine, busulfan, procarbazine, and melphalan.

As used in the present disclosure, the term “combination,” “combined,” or a variation thereof is intended to define a therapy involving the use of two or more compound/drug combinations. The term can refer to compounds/drugs that are administered as part of the same overall dosage schedule. The respective dosages of two or more compounds/drugs can be different. The combination therapy is intended to embrace administration of these compounds/drugs in a sequential manner, that is, wherein each compound/drug is administered at a different time, as well as administration of these compounds/drugs, or at least two of the compounds/drugs, in a substantially simultaneous manner. Substantially simultaneous administration can be accomplished, for example, by administering to the subject a single dosage form having a fixed ratio of each compound/drug or in multiple, single dosage forms for each of the compounds/drugs. Sequential or substantially simultaneous administration of each compound/drug can be effected by any appropriate route including, but not limited to, oral routes, intravenous routes, intramuscular routes, and direct absorption through mucous membrane tissues (e.g., buccal). The compounds/drugs can be administered by the same route or by different routes. For example, a first compound/drug of the combination selected may be administered by intravenous injection while the other compound/drug of the combination may be administered orally. Alternatively, for example, all compounds/drugs may be administered orally or all compounds/drugs may be administered by intravenous injection.

Combination therapy also can embrace the administration of the compounds/drugs as described above in further combination with other biologically active ingredients and non-drug therapies (e.g., surgery or radiation treatment). Where the combination therapy further comprises a non-drug treatment, the non-drug treatment may be conducted at any suitable time so long as a beneficial effect from the co-action of the combination of compound/drug and non-drug treatment is achieved. For example, in appropriate cases, the beneficial effect is still achieved when the non-drug treatment is temporally removed from the administration of the compound/drug, perhaps by days or even weeks.

### EXAMPLES

Study Design. An open-label, nonrandomized, Phase 2 study of Compound (1) was performed on approximately 100 patients with iCCA harboring confirmed FGFR2 gene fusions or other FGFR2 rearrangements who have failed all standard therapies or for whom standard therapy does not exists or is not tolerated.

Inclusion Criteria. Patients were administered treatment with Compound (1) that met all of the following inclusion criteria:
1) Provided written informed consent form (ICF).
2) ≥18 years of age
3) Had histologically or cytologically confirmed, locally advanced, metastatic cancer that met the following criteria:
   (i) histologically or cytologically confirmed, locally advanced, metastatic, unresectable iCCA harboring FGFR2 gene fusions or other FGFR2 rearrangements based on results from either of the following:
      a. Testing by Foundation Medicine:
         i. As part of study pre-screening; or
         ii. Previously tested by Foundation Medicine;
      b. Local laboratory testing using next generation sequencing [NGS], fluorescence in situ hybridization [FISH], or other assays that can determine FGFR2 gene fusions or other FGFR2 rearrangements on tumor tissues or from ctDNA;
   (ii) Patient was treated with at least one prior systemic gemcitabine and platinum-based chemotherapy. Patients with prior adjuvant gemcitabine-platinum chemotherapy were eligible if the patient had recurrence within 6 months of the last dose of the regimen
   (iii) Patient had documentation of radiographic disease progression on the most recent prior therapy
4) Patient had measurable disease as defined by Response Evaluation Criteria in Solid Tumors (RECIST) guidelines (version 1.1, 2009) for advanced solid tumors.
5) Eastern Cooperative Oncology Group (ECOG) performance status 0 or 1 on Day 1 of Cycle 1.
6) Able to take medications orally (e.g., no feeding tube).

Exclusion Criteria. Patients were excluded from treatment that were treated with any of the following within the specified time frame prior to first dose of Compound (I):
1) Major surgery within the previous 4 weeks (the surgical incision should be fully healed prior to treatment with Compound (1)).
2) Radiotherapy for extended field within 4 weeks or limited field radiotherapy within 2 weeks.
3) Patients with locoregional therapy, e.g., transarterial chemoembolization (TACE), selective internal radiotherapy (SIRT) or ablation within 4 weeks.
4) Any noninvestigational anticancer therapy within 3 weeks or have not recovered from side effects of such therapy prior to Compound (1) administration (mitomycin within prior 5 weeks).
   Targeted therapy or immunotherapy within 3 weeks or within 5 halflives (whichever is shorter)
5) Any investigational agent received within 5 half-lives of the drug or 4 weeks, whichever is shorter. Concurrent participation in an observational study may be allowed.
6) Patients with prior FGFR-directed therapy.

Study Drug Administration. Futibatinib (“Compound (1)”—(S)-1-[(3)-[4-amino-3-[(3,5-dimethoxyphenyl)ethynyl]-1H-pyrazolo[3,4-d]pyrimidin-1-yl]-1-pyrrolidinyl]-2-propen-1-one—was supplied as 4 mg film-coated tablets. Film-coated tablets of Compound (1) were formulated using sodium lauryl sulfate, lactose monohydrate, corn starch, low viscosity hydroxypropyl cellulose, D-mannitol, microcrystalline cellulose, crospovidone, and magnesium stearate as the carrier system, and hypromellose, polyethylene glycol, titanium oxide, and coloring agent for the coating, according to US2021/0030755 and WO2019/181876, the contents of which are incorporated herein by reference in their entirety. The dose for Compound (1) was 20 mg QD. Patients were required to fast for at least 2 hours before and 1 hour after administration of Compound (1), but were permitted to drink water during this period. If a patient missed a dose (i.e., did not take Compound (1) for >12 hours of the scheduled time of that day), the patient was instructed to take the dose on the next day.

Treatment Regimen. Compound (1) was administered orally as a daily, continuous, 21-day treatment cycle until at least 1 of the following was met; disease progression, unacceptable adverse events (AEs), withdrawal of consent, or death. There were no breaks in dosing between cycles. A maximum of two dose reductions were permitted if AEs were observed. For a first dose reduction, the dose was reduced to 16 mg QD. For a second dose reduction, the dose was reduced to 16 mg QD.

Tumor Assessments. Tumor assessments/imaging studies of the chest, abdomen, and pelvis (as clinically indicated) were obtained at each time point listed below for all patients with solid tumors:
Screening within 28 days prior to Day 1 of Cycle 1. Computed tomography scans obtained prior to the signed ICF may be used as the screening scan if they were obtained within 28 days of the first dose of Compound (1).
At the end of every 2 cycles (up to +2 weeks), up to Cycle 4
Following Cycle 4, at least after every 3 cycles (+7 days) or as clinically indicated, until documented progression (including after end of treatment if the patient discontinues for reasons other than radiologic disease progression).
At end of treatment (+0-7 days), a CT scan was performed if the prior scan was performed ≥9 weeks prior to discontinuation of Compound (1) treatment if the patient discontinued for reasons other than radiologic disease progression.

On-site tumor assessments were performed by the investigator/local radiologist according to RECIST guidelines (version 1.1, 2009). Results of these assessments, including response for target and non-target lesions and appearance of new lesions, were the basis for the continuation or discontinuation of treatment with Compound (1).

Efficacy Assessment. Tumor assessments were performed as indicated above. The determination of antitumor efficacy was based on objective tumor assessments made by the investigator according to the revised RECIST guidelines (version 1.1, 2009) of unidimensional evaluation. The primary endpoint was Objective Response Rate (ORR) and the secondary endpoints were Duration of response (DOR), Disease control rate (DCR), Progression-free survival (PFS), Patient Reported Outcomes (PROs), and Overall Survival (OS) (response evaluations based on independent review of images by the Core Imaging Laboratory). In addition, sensitivity analyses for some key efficacy endpoints (notably ORR and PFS) were performed based on assessments by the investigator or local radiologist.

Objective Response Rate (ORR). Objective response rate (ORR) is defined as the proportion of patients with objective evidence of complete response (CR) or partial response (PR). CR is defined as the disappearance of all target lesions (Any pathological lymph nodes must have reduction in short axis to <10 mm). PR is defined as at least a 30% decrease in the sum of diameters of the target lesions, taking as a reference the baseline sum diameters. The number sum of patients determined to have a CR or PR is referred to as “Responders.” Thus, when expressed as a percentage, ORR is the number of responders (n) per the total number of patients in the group (e.g., with the same genomic alteration). The evaluation of ORR was based on investigator assessment and/or central independent review of the images as follows:
Central independent CT/MRI image assessments (primary analysis) and local CT/MRI image assessments (sensitivity analysis).
   At the analysis stage, the best objective response was assigned for each patient as the best response recorded after initiation of study treatment and confirmed at least 4 weeks later. If applicable, responses recorded after disease progression or initiation of new anticancer treatment were excluded. The exact 2-sided CI based on Clopper-Pearson methodology was derived for ORR.

Progression-free survival (PFS). Progression-free survival (PFS) is defined as the time from the day of the first dose to the date of first objectively documented disease progression or death (any cause), whichever occurs first. Patients who die without a reported disease progression were considered to have progressed on the date of their death. Patients who did not progress or die were censored on the date of their last tumor assessment. Patients who did not have any on-study assessments and did not die were censored on the first dosing date. Patients who started any subsequent anti-cancer therapy without a prior reported progression were censored at the last tumor assessment prior to initiation of the subsequent anti-cancer therapy. Progression-free survival was also analyzed as a time-to-event endpoint with the median (Kaplan-Meier estimate) and associated 95% CI (Brookmeyer-Crowley methodology) reported, along with the Kaplan-Meier estimates for PFS rates at 3, 6, 9 and 12 months and associated 95% CIs (log-log transformation methodology of Kalbfleisch-Prentice).

Subanalysis by co-occurring genetic alterations. Subanalysis was performed on patients with FGFR2 rearrangements/fusions who also had a co-occurring genetic alteration in at least one cancer driver gene. Cancer driver gene alteration was histologically or cytologically confirmed using the same methods used for determining FGFR2 genetic alterations.

Results. Subjects with genomic alteration data who had FGFR2 fusions/rearrangements treated with Compound (1) according to the above were subanalyzed according to co-occurring genomic alterations in various cancer driver genes. The results with respect to ORR and median PFS are presented in Table 1.

TABLE 1
Summary of Genomic Alteration by Variants

| | Numbers of | | | | | |
|---|---|---|---|---|---|---|
| | subjects | | | ORR | Median | |
| Genomic | with Genomic | Responders | | 95% | PFS (month) | |
| Alterations^{a} | Alterations | n (%)^{b} | | CI (%) | 95% CI | |
| FGFR2 | | | | | | |
| unaltered | 0 | 0 | (0.0%) | | | |
| altered | 93 | 40 | (43.0%) | (32.8, 53.7) | 8.9 | (6.6, 13.1) |
| rearrangement | 93 | 40 | (43.0%) | (32.8, 53.7) | 8.9 | (6.6, 13.1) |
| short-variant | 3 | 1 | (33.3%) | (0.8, 90.6) | 8.9 | (4.6, 9.7) |
| BAP1 | | | | | | |
| unaltered | 53 | 26 | (49.1%) | (35.1, 63.2) | 8.0 | (4.9, 13.8) |
| altered | 40 | 14 | (35.0%) | (20.6, 51.7) | 9.0 | (5.1, 13.3) |
| copy-number-alteration | 2 | 1 | (50.0%) | (1.3, 98.7) | 13.3 | (NE, NE) |
| rearrangement | 1 | 0 | (0.0%) | | 1.2 | (NE, NE) |
| short-variant | 37 | 13 | (35.1%) | (20.2, 52.5) | 9.0 | (5.1, 13.1) |
| CDKN2A | | | | | | |
| unaltered | 73 | 32 | (43.8%) | (32.2, 55.9) | 9.7 | (6.9, 13.8) |
| altered | 20 | 8 | (40.0%) | (19.1, 63.9) | 4.9 | (3.4, 13.3) |
| copy-number-alteration | 17 | 7 | (41.2%) | (18.4, 67.1) | 4.8 | (3.2, 6.1) |
| rearrangement | 1 | 0 | (0.0%) | | 2.8 | (NE, NE) |
| short-variant | 3 | 1 | (33.3%) | (0.8, 90.6) | NE | (8.9, NE) |
| CDKN2B | | | | | | |
| unaltered | 77 | 33 | (42.9%) | (31.6, 54.6) | 11.0 | (7.2, 15.1) |
| altered | 16 | 7 | (43.8%) | (19.8, 70.1) | 4.8 | (3.4, 4.9) |
| copy-number-alteration | 15 | 7 | (46.7%) | (21.3, 73.4) | 4.8 | (2.8, 6.1) |
| short-variant | 1 | 0 | (0.0%) | | 4.9 | (NE, NE) |
| TP53 | | | | | | |
| unaltered | 80 | 35 | (43.8%) | (32.7, 55.3) | 9.0 | (6.6, 13.3) |
| altered | 13 | 5 | (38.5%) | (13.9, 68.4) | 7.0 | (1.4, 13.8) |
| short-variant | 13 | 5 | (38.5%) | (13.9, 68.4) | 7.0 | (1.4, 13.8) |
| ARID1A | | | | | | |
| unaltered | 81 | 34 | (42.0%) | (31.1, 53.5) | 9.0 | (6.2, 13.1) |
| altered | 12 | 6 | (50.0%) | (21.1, 78.9) | 8.8 | (4.9, 18.2) |
| short-variant | 12 | 6 | (50.0%) | (21.1, 78.9) | 8.8 | (4.9, 18.2) |
| MLL2 | | | | | | |
| unaltered | 82 | 33 | (40.2%) | (29.6, 51.7) | 8.9 | (6.2, 13.1) |
| altered | 11 | 7 | (63.6%) | (30.8, 89.1) | 9.0 | (2.7, NE) |
| short-variant | 11 | 7 | (63.6%) | (30.8, 89.1) | 9.0 | (2.7, NE) |
| PIK3C2B | | | | | | |
| unaltered | 82 | 33 | (40.2%) | (29.6, 51.7) | 8.9 | (6.0, 13.1) |
| altered | 11 | 7 | (63.6%) | (30.8, 89.1) | 9.0 | (4.7, 15.2) |
| copy-number-alteration | 10 | 6 | (60.0%) | (26.2, 87.8) | 9.0 | (1.4, 15.2) |
| rearrangement | 1 | 0 | (0.0%) | | 1.4 | (NE, NE) |
| short-variant | 2 | 2 | (100%) | (15.8, 100.0) | 6.4 | (4.7, 8.0) |
| IKBKE | | | | | | |
| unaltered | 83 | 34 | (41.0%) | (30.3, 52.3) | 8.9 | (5.1, 13.3) |
| altered | 10 | 6 | (60.0%) | (26.2, 87.8) | 9.3 | (6.1, 11.3) |
| copy-number-alteration | 7 | 5 | (71.4%) | (29.0, 96.3) | 9.0 | (6.1, 11.3) |
| short-variant | 5 | 2 | (40.0%) | (5.3, 85.3) | 9.7 | (7.2, 9.7) |
| MCL1 | | | | | | |
| unaltered | 83 | 35 | (42.2%) | (31.4, 53.5) | 8.9 | (6.0, 11.3) |
| altered | 10 | 5 | (50.0%) | (18.7, 81.3) | 13.1 | (1.4, 13.1) |
| copy-number-alteration | 10 | 5 | (50.0%) | (18.7, 81.3) | 13.1 | (1.4, 13.1) |
| MDM4 | | | | | | |
| unaltered | 83 | 34 | (41.0%) | (30.3, 52.3) | 8.9 | (6.0, 13.1) |
| altered | 10 | 6 | (60.0%) | (26.2, 87.8) | 9.0 | (1.4, 15.2) |
| copy-number-alteration | 10 | 6 | (60.0%) | (26.2, 87.8) | 9.0 | (1.4, 15.2) |
| short-variant | 1 | 1 | (100%) | (2.5, 100.0) | 6.9 | (NE, NE) |
| MTAP | | | | | | |
| unaltered | 83 | 35 | (42.2%) | (31.4, 53.5) | 9.0 | (6.7, 13.1) |
| altered | 10 | 5 | (50.0%) | (18.7, 81.3) | 4.9 | (1.4, NE) |
| copy-number-alteration | 10 | 5 | (50.0%) | (18.7, 81.3) | 4.9 | (1.4, NE) |
| MYC | | | | | | |
| unaltered | 83 | 35 | (42.2%) | (31.4, 53.5) | 8.9 | (6.6, 11.3) |
| altered | 10 | 5 | (50.0%) | (18.7, 81.3) | 10.8 | (2.6, NE) |
| copy-number-alteration | 10 | 5 | (50.0%) | (18.7, 81.3) | 10.8 | (2.6, NE) |
| PBRM1 | | | | | | |
| unaltered | 84 | 38 | (45.2%) | (34.3, 56.5) | 9.0 | (6.7, 13.1) |
| altered | 9 | 2 | (22.2%) | (2.8, 60.0) | 4.8 | (1.2, 16.7) |
| copy-number-alteration | 2 | 1 | (50.0%) | (1.3, 98.7) | 16.7 | (NE, NE) |
| rearrangement | 2 | 1 | (50.0%) | (1.3, 98.7) | 3.0 | (1.2, 4.9) |
| short-variant | 5 | 0 | (0.0%) | | 3.4 | (1.4, 4.8) |
| PTEN | | | | | | |
| unaltered | 90 | 40 | (44.4%) | (34.0, 55.3) | 9.0 | (6.7, 13.1) |
| altered | 3 | 0 | (0.0%) | | 4.6 | (1.4, 4.8) |
| copy-number-alteration | 1 | 0 | (0.0%) | | 1.4 | (NE, NE) |
| short-variant | 2 | 0 | (0.0%) | | 4.7 | (4.6, 4.8) |

^{a}Genes with more than one variant are counted in each variant category
^{b}Denominator is the number of patients with the same genomic alteration
NE = not estimated

Comparative results with pemigatinib. Table 2 shows previously reported results (ORR and median PFS) of patients with co-occurring genomic alterations in FGFR2 and various cancer driver genes treated with pemigatinib. Overall, patients harboring a co-occurring genetic alteration in a tumor suppressor gene, including BAP1, CDKN2A/B, PBRM1, TP53, ARID1A, and PTEN, had significantly shorter median PFS (6.8 months) than those with unaltered tumor suppressor genes (11.7 months). From the data of individual tumor suppressor genes presented, patients with alterations in CDKN2A B, PBRM1 and TP53 had significantly shorter median PFS on pemigatinib than those without alterations in these genes. In particular, subjects with a co-occurring TP53 alteration had an ORR of 0% and a significantly reduced median PFS of 2.8 months, versus an ORR of 38.8% and median PFS of 9.0 months when TP53 was unaltered. While not as significant, alterations to BAP1 also trended to provide shorter median PFS in the altered state (6.9 months versus 9.1 months unaltered). This trend was also seen for oncogenes PIK3CA and IDH1, which showed shorter median PFS in the altered state compared to when these genes were unaltered.

TABLE 2
(Comparative) Previously reported results
from treatment with pemigatinib^{a,b}

| | Numbers of | | | |
|---|---|---|---|---|
| | subjects with | | Median | |
| Genomic | Genomic | ORR | PFS (month) | |
| Alterations | Alterations | (%) | 95% CI | |
| Tumor suppressor^{c} | | | | |
| unaltered | 43 | 37.2 | 11.7 | (9.1-17.4) |
| altered | 64 | 34.4 | 6.8 | (4.9-6.9) |
| BAP1 | | | | |
| unaltered | 68 | 30.9 | 9.1 | (6.2-11.7) |
| altered | 39 | 43.6 | 6.9 | (4.7-8.9) |
| CDKN2A/B | | | | |
| unaltered | 86 | 38.4 | 9.0 | (6.4-11.1) |
| altered | 21 | 23.8 | 6.4 | (1.7-6.9) |
| PBRM1 | | | | |
| unaltered | 97 | 36.1 | 7.0 | (6.8-10.5) |
| altered | 10 | 30.0 | 4.7 | (1.4-10.8) |
| TP53 | | | | |
| unaltered | 98 | 38.8 | 9.0 | (6.8-11.1) |
| altered | 9 | 0 | 2.8 | (1.4-6.8) |
| PIK3CA | | | | |
| unaltered | 98 | 35.7 | 8.8 | (6.4-10.5) |
| altered | 9 | 33.3 | 5.2 | (1.5-11.1) |
| IDH1 | | | | |
| unaltered | 102 | 36.3 | 6.9 | (6.1-9.6) |
| altered | 5 | 20.0 | NE | (1.4-NE) |

^{a}Data reported in Silverman IM, Hollebecque A, Friboulet L, et al. Clinicogenomic Analysis of FGFR2-Rearranged Cholangiocarcinoma Identifies Correlates of Response and Mechanisms of Resistance to Pemigatinib. Cancer Discov February 2021 (11) (2) 326-339
^{b}Data based on 107 patients with cholangiocarcinoma harboring FGFR2 rearrangements/fusions
^{c}data includes BAP1, CDKN2A/B, PBRM1, TP53, ARID1A, and PTEN
NE = not estimated

Analysis of Compound (1). In contrast to the findings with pemigatinib, subjects treated with Compound (1) that had a co-occurring genetic alteration of FGFR2 and a tumor suppressor gene TP53, BAP1, and ARID1A were surprisingly found to respond as well or similarly to treatment as those without alteration to the tumor suppressor gene. The results obtained from subjects with TP53 alterations were particularly striking, with values for ORR and median PFS of 38.5% and 7 months, respectively, being nearly comparable to 43.8% and 9 months for subjects with unaltered TP53—whereas pemigatinib appeared to be ineffective at treating this patient population. Subjects with alterations to other tumor suppressor genes such as MLL2 were also surprisingly identified as responders to treatment with Compound (1). Also unexpected was the finding that subjects having a co-occurring genetic alteration to oncogenes PIK3C2B, IKBKE, MCL1, MDM4, and MYC were positive responders to treatment with Compound (1), with ORR and median PFS values that were actually higher than those found in subjects without alterations to these oncogenes.

Obviously, numerous modifications and variations of the present invention are possible in light of the above teachings. It is therefore to be understood that within the scope of the appended claims, the invention may be practiced otherwise than as specifically described herein.

1. A method of treating a subject with cholangiocarcinoma having a co-occurring genetic alteration in FGFR2 and a cancer driver gene selected from the group consisting of TP53, BAP1, ARID1A, MLL2, PIK3C2B, IKBKE, MCL1, MDM4, and MYC, the method comprising:
administering to the subject an effective amount of (S)-1-[(3)-[4-amino-3-[(3,5-dimethoxyphenyl)ethynyl]-1H-pyrazolo[3,4-d]pyrimidin-1-yl]-1-pyrrolidinyl]-2-propen-1-one or a pharmaceutically acceptable salt thereof.

2. The method of claim 1, wherein the genetic alteration in FGFR2 is a FGFR2 rearrangement or fusion.

3. The method of claim 1, wherein the genetic alteration in FGFR2 is a FGFR2 rearrangement.

4. The method of claim 1, wherein the genetic alteration in FGFR2 is a FGFR2 fusion.

5. The method of claim 4, wherein the FGFR2 fusion is selected from the group consisting of FGFR2-ARHGAP22, FGFR2-AXDND1, FGFR2-AZI1, FGFR2-BEND3, FGFR2-BFSP2, FGFR2-BICC1, FGFR2-CA10, FGFR2-CCDC147, FGFR2-CEP44, FGFR2-CEP55, FGFR2-CIT, FGFR2-CREB5, FGFR2-CTNNA3, FGFR2-CUX1, FGFR2-DDX21, FGFR2-EVI5, FGFR2-GPHN, FGFR2-INA, FGFR2-KIAA1217, FGFR2-KIAA1524, FGFR2-KIAA1598, FGFR2-LRBA, FGFR2-MACF1, FGFR2-MYH9, FGFR2-NRBF2, FGFR2-OFD1, FGFR2-PDE3B, FGFR2-POC1B, 1, FGFR2-PUM1, FGFR2-RBM20, FGFR2-RXRG, FGFR2-SEC21IP, FGFR2-SH3KBP1, FGFR2-SHROOM3, FGFR2-SLMAP, FGFR2-SMARCC1, FGFR2-SORBS1, FGFR2-SYNPO2, FGFR2-TACC1, FGFR2-TACC2, FGFR2-TBC1D4, FGFR2-TRIM8, FGFR2-TUFT1, FGFR2-TXLNA, FGFR2-VCL, and FGFR2-WAC.

6. The method of claim 4, wherein the FGFR2 fusion is selected from the group consisting of FGFR2-ARHGAP22, FGFR2-AXDND1, FGFR2-BEND3, FGFR2-BFSP2, FGFR2-BICC1, FGFR2-CCDC147, FGFR2-CIT, FGFR2-CTNNA3, FGFR2-CUX1, FGFR2-DDX21, FGFR2-GPHN, FGFR2-KIAA1217, FGFR2-KIAA1524, FGFR2-KIAA1598, FGFR2-MACF1, FGFR2-PDE3B, FGFR2-RBM20, FGFR2-RXRG, FGFR2-SH3KBP1, FGFR2-SMARCC1, FGFR2-TACC1, FGFR2-TACC2, FGFR2-TUFT1, and FGFR2-VCL.

7. The method of claim 4, wherein the FGFR2 fusion is selected from the group consisting of FGFR2-BICC1, FGFR2-KIAA1217, and FGFR2-SMARCC1.

8. The method of claim 1, wherein the cancer driver gene is selected from the group consisting of TP53, BAP1, and ARID1A.

9. The method of claim 1, wherein the cancer driver gene is selected from the group consisting of BAP1, ARID1A, MLL2, PIK3C2B, IKBKE, MCL1, MDM4, and MYC.

10. The method of claim 1, wherein the cancer driver gene is selected from the group consisting of BAP1 and ARID1A.

11. The method of claim 1, wherein the cancer driver gene is TP53.

12. The method of claim 11, wherein the genetic alteration in TP53 is a short-variant mutation.

13. The method of claim 1, wherein the cancer driver gene is BAP1.

14. The method of claim 13, wherein the genetic alteration in BAP1 is a short-variant mutation or a copy-number alteration.

15. The method of claim 1, wherein the cancer driver gene is ARID1A.

16. The method of claim 15, wherein the genetic alteration in ARID1A is a short-variant mutation.

17. The method of claim 1, wherein the cancer driver gene is MLL2.

18. The method of claim 17, wherein the genetic alteration in MLL2 is a short-variant mutation.

19. The method of claim 1, wherein the cancer driver gene is PIK3C2B.

20. The method of claim 19, wherein the genetic alteration in PIK3C2B is a short-variant mutation or a copy-number alteration.

21. The method of claim 1, wherein the cancer driver gene is IKBKE.

22. The method of claim 21, wherein the genetic alteration in IKBKE is a short-variant mutation or a copy-number alteration.

23. The method of claim 1, wherein the cancer driver gene is MCL1.

24. The method of claim 23, wherein the genetic alteration in MCL1 is a copy-number alteration.

25. The method of claim 1, wherein the cancer driver gene is MDM4.

26. The method of claim 25, wherein the genetic alteration in MDM4 is a short-variant mutation or a copy-number alteration.

27. The method of claim 1, wherein the cancer driver gene is MYC.

28. The method of claim 27, wherein the genetic alteration in MYC is a copy-number alteration.

29. The method of claim 1, wherein the subject with cholangiocarcinoma is determined to have the co-occurring genetic alteration in FGFR2 and the cancer driver gene prior to the administering.

30. The method of claim 1, wherein the cholangiocarcinoma is intrahepatic cholangiocarcinoma.

31. The method of claim 1, wherein the cholangiocarcinoma is extrahepatic cholangiocarcinoma.

32. The method of claim 1, wherein the cholangiocarcinoma is unresectable.

33. The method of claim 1, wherein the subject with cholangiocarcinoma has previously undergone a chemotherapy regimen prior to the administering.

34. The method of claim 1, wherein the subject with cholangiocarcinoma has previously undergone a chemotherapy regimen with at least one selected from the group consisting of gemcitabine, cisplatin, fluorouracil, leucovorin, and oxaliplatin, prior to the administering.

35. The method of claim 1, wherein the (S)-1-[(3)-[4-amino-3-[(3,5-dimethoxyphenyl)ethynyl]-1H-pyrazolo[3,4-d]pyrimidin-1-yl]-1-pyrrolidinyl]-2-propen-1-one or a pharmaceutically acceptable salt thereof is administered orally to the subject.

36. The method of claim 1, wherein the (S)-1-[(3)-[4-amino-3-[(3,5-dimethoxyphenyl)ethynyl]-1H-pyrazolo[3,4-d]pyrimidin-1-yl]-1-pyrrolidinyl]-2-propen-1-one or a pharmaceutically acceptable salt thereof is administered to the subject once per day (QD).

37. The method of claim 1, wherein 1 to 20 mg of (S)-1-[(3)-[4-amino-3-[(3,5-dimethoxyphenyl)ethynyl]-1H-pyrazolo[3,4-d]pyrimidin-1-yl]-1-pyrrolidinyl]-2-propen-1-one or a pharmaceutically acceptable salt thereof is administered to the subject per day.

38. The method of claim 1, wherein the (S)-1-[(3)-[4-amino-3-[(3,5-dimethoxyphenyl)ethynyl]-1H-pyrazolo[3,4-d]pyrimidin-1-yl]-1-pyrrolidinyl]-2-propen-1-one or a pharmaceutically acceptable salt thereof is administered daily to the subject for at least 21 days.
